(19) United States
(12) Patent Application Publication
(10) Pub. No.: US 2025/0054586 A1
(43) Pub. Date: Feb. 13, 2025

(54) ELECTRONIC GLOBAL PERSONAL HEALTH RECORDS SYSTEM

(71) Applicant: One Clinic Company Limited, Ho Chi Min City (VN)

(72) Inventors: Francis Tuan Anh Nguyen, San Jose, CA (US); Ut Van Huynh, San Jose, CA (US)

(73) Assignee: One Clinic Company Limited, ho Chi Minh City (VN)

(21) Appl. No.: 18/232,253

(22) Filed: Aug. 9, 2023

(51) Int. Cl. G16H 10/60; H04L 9/00

(52) U.S. Cl. CPC G16H 10/60; H04L 9/50

(57) ABSTRACT

The system described herein is a Global Personal Health Records (PHR) system that supports: 1) retrieving and storing data offline in HL7 FHIR local storage and online in HL7 FHIR server for the entire household, each member having their own records; 2) retrieving and storing data from user inputs (handwriting or voice), from hospital systems of records (EHR, or EMR and HIS/CIS), from personal health/medical devices (Apple HealthKit, Blood Pressure Monitor, Continuous Glucose Monitor, etc.) via PHR Device Hub (OneHub), and from insurance reimbursement and authorization systems; 3) data mining for descriptive analytics like real-time patient monitoring (RPM), for predictive analytics like drug interaction warnings, or for prescriptive analytics such as exam or lab recommendations; and 4) secured data sharing using a Blockchain protected system.

### BACKGROUND

### Field of the Invention

The present disclosure relates to the digital transformation of healthcare systems, and more particularly to a system for transforming paper-based personal health records (paper and film) to electronic personal health records (PHR).

### Description of the Related Art

A patient's medical records are often contained in multiple paper-based form template(s) (e.g., physician's diagnosis, laboratory test results, medicine prescriptions, etc.) and films (e.g., x-rays, MRI, etc.). Healthcare Professionals (HCP) must have access to all these paper forms and films to provide effective treatments. Patients must have access to these paper forms and films to follow their diagnosis and treatments.

Most existing Personal Health Records (PHR) systems retrieve and store data using proprietary formats. Using proprietary formats is not as efficient and portable as using HL7 (Health Level 7) FHIR (Fast Healthcare Interoperability Resources) formatted records to retrieve and store data offline in HL7 FHIR local storage and online in HL7 FHIR servers. FHIR resources are controlled by a global standard and are therefore application independent. HL7 FHIR data can be shared across multiple applications or systems, located in one or multiple countries, without any need for customization or change to the database.

Personal health/medical devices are often bundled with manufacturer applications which retrieve and store data to local storage and/or private servers without sharing the data to any of the other databases. Accordingly, patients have limited ability to share their own healthcare data with health care professionals. Authentication methods using digital signatures are expensive and difficult to scale up.

### SUMMARY

The present disclosure describes an electronic PHR system that supports retrieving and storing data offline in HL7 FHIR formatted local storage and online in HL7 FHIR formatted servers. The present invention supports replacing the data fields in a given PHR with data tags that can be used to retrieve data from user inputs, from hospital records systems (EHR (Electronic Health Records), or EMR (Electronic Medical Records) and HIS/CIS (Hospital Information Systems/Clinical Information Systems), from personal health/medical devices via a PHR device hub (OneHub or the like), and from insurance reimbursement and authorization systems.

Using the system disclosed herein, health care professionals can import the patient's historical data from the PHR to their hospital systems of records for first-time patient visits. The health care professional can then share the examination data of the patient from their hospital systems of record storage to the PHR after the patient visit.

The presently disclosed system allows family members and/or designated representatives (such as a head of household) to connect personal health/medical devices to PHR device hubs (e.g. OneHub) via Ethernet, Wi-Fi, BLE (Bluetooth Low Energy) or Serial protocols. Then the devices can auto push personal health/medical data to selected PHR systems.

The presently disclosed system retrieves and stores all health records as local storage HL7 FHIR records so that patient data can be quickly and efficiently data mined for descriptive analytics such as real-time patient monitoring (RPM) based on vital sign data from personal health/medical devices worn by the patient, predictive analytics such as drug interaction warnings based on history of treatment, diagnosis, prescription, lab results from health facilities visited by the patient, and prescriptive analytics like medical exams or lab test recommendations based on big data of medical treatments of current medical conditions experienced by the patient.

The presently disclosed system further allows the PHR data to be securely shared between patients and health care professionals using a Blockchain based system. The method may also include, upon signing the subject document, generating a digitized image including images of the one or more electronic signatures. The method may then proceed with generating a hash based on the digitized image. The method may also include storing the signed form in a data repository. The method may then proceed with storing the hash to a blockchain system. The hash can be used to verify authenticity of the signed form.

### BRIEF DESCRIPTION OF THE DRAWINGS

FIG. 1 is a block diagram illustrating the initiation of records in the Global Personal Health Records (GPHR) system (alternatively referred to herein as the OnePHR system).

FIG. 2A is a flowchart illustrating the process flow of a patient establishing a PHR account in the GPHR system.

FIG. 2B is a flowchart illustrating the various steps of a patient being evaluated for medical testing.

FIG. 2C is a flowchart illustrating the process required for a patient being evaluated for a prescription.

FIG. 2D is a flowchart illustrating the processing of an insurance claim in the GPHR system.

FIG. 3 illustrates the process for an HCP to request access to a PHR account and the mechanism for a patient to grant access to the PHR account.

FIG. 4 summarizes the various data flows within the GPHR system.

FIG. 5 is a high-level block diagram illustrating an example computer system, within which a set of instructions for causing a machine to perform any one or more of the methodologies discussed herein can be executed.

### DETAILED DESCRIPTION

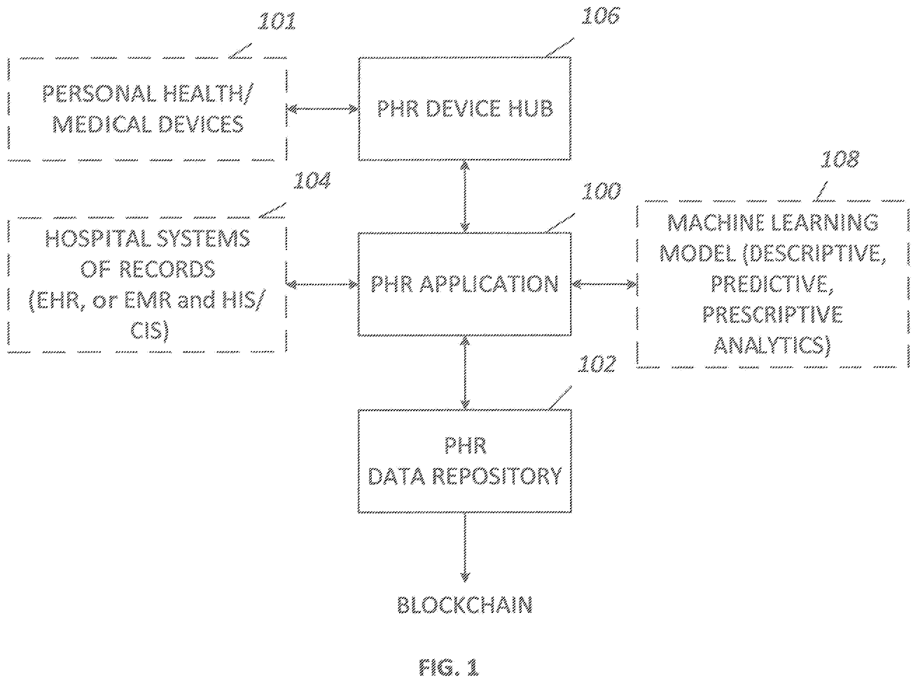

FIG. 1 illustrates the architecture of the system presently disclosed, referred to herein as the GPHR system or alternatively, the OnePHR system. The process is initiated by healthcare professionals or patients uploading patient data to a PHR application 100. The PHR application 100 stores the patient data in a PHR data repository 102. With appropriate authorization, the patient data is accessible by medical professionals in the PHR data repository 102. The PHR application 100 is also in communication with a hospital system of records 104. This communication link enables medical professionals to retrieve a patient's medical history, diagnoses, medications, treatment plans, immunization dates, allergies, radiology images, and laboratory and test results. The PHR application 100 retrieves patient data in real-time at the point of care, the point of care being the location where healthcare providers perform medical examinations and provide treatment.

The PHR application 100 communicates with a PHR device hub 106 to receive, store, and enable retrieval of health and medical data from personal health/medical devices. The PHR data repository 102 transfers patient data from the PHR system to other healthcare providers via a Blockchain-secured interface. As the PHR application 100 retrieves patient data, the application 100 may trigger a machine learning model 108 to analyze the data for descriptive purposes (e.g., visualizing vital signs for remote patient monitoring based on vital sign data from personal health/medical devices worn by the patient), predictive purposes (e.g., warning drug interaction based on history of treatment, diagnosis, prescription, lab results from health facilities visited by the patient), and/or prescriptive purposes (e.g., recommending specific medical exams or lab tests based on big data of medical treatments of current medical conditions experienced by the patient).

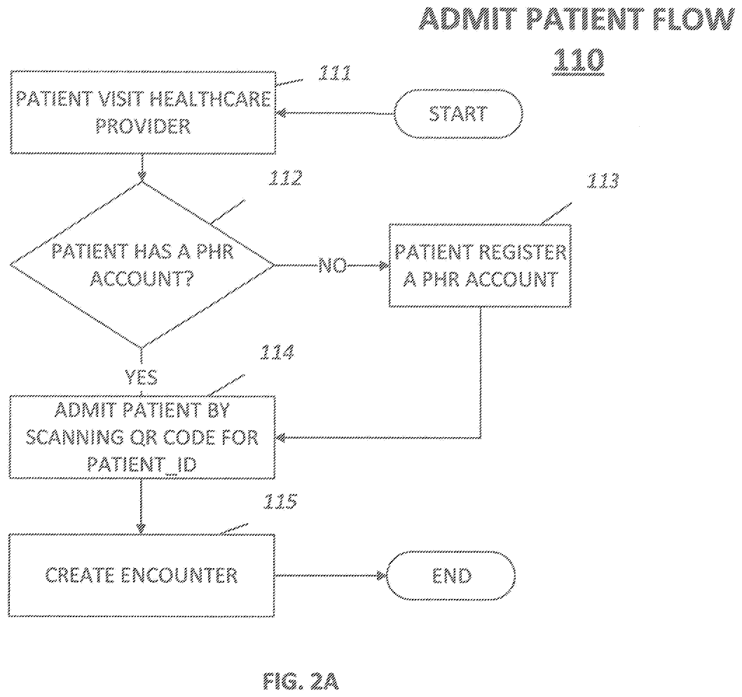

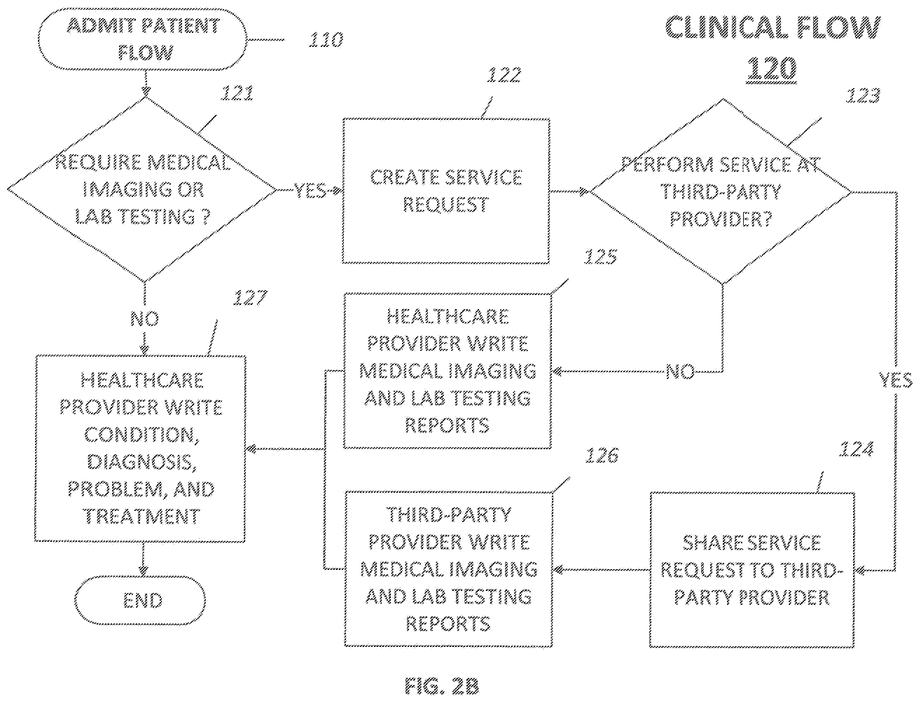

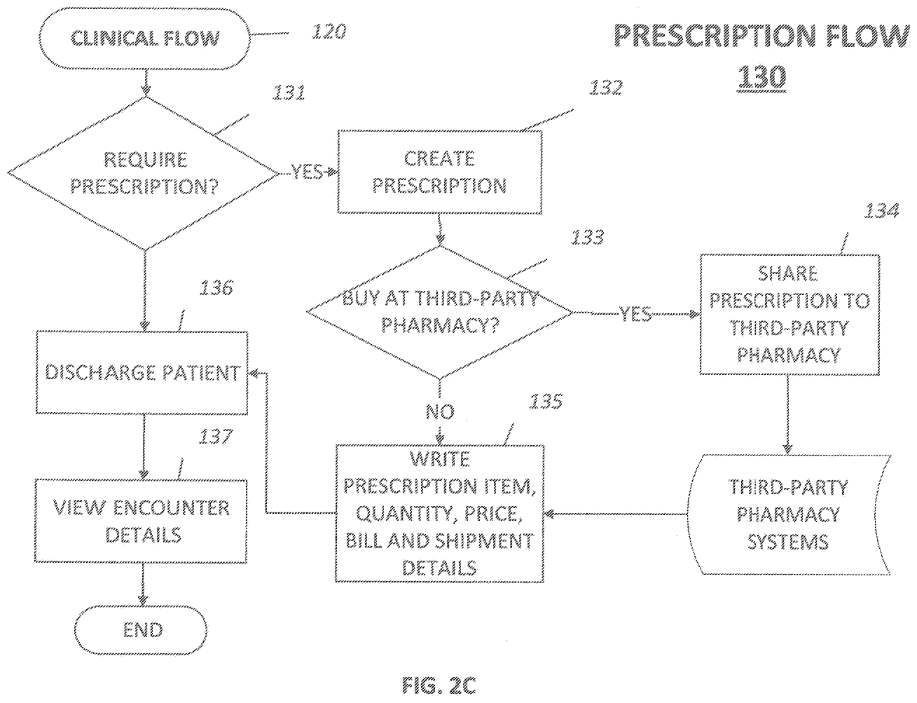

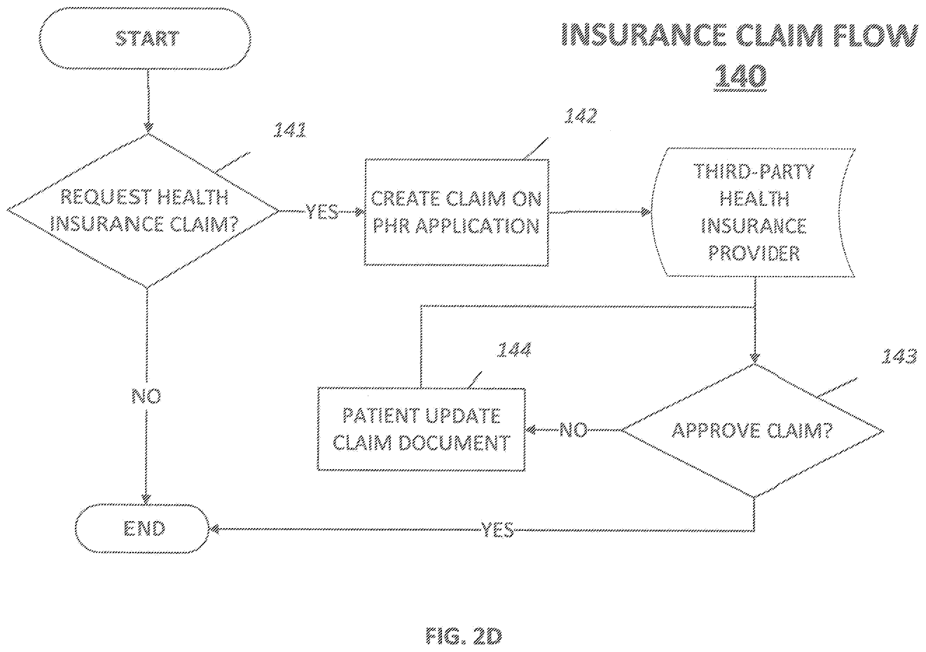

FIGS. 2A-D are flow charts illustrating the process followed by a patient registering for a PHR account and an HCP adding data to the PHR account using the PHR system. FIG. 2A illustrates the initialization step 110 of the process by a patient visiting the office of an HCP in step 111. Next, in step 112, the HCP checks to determine whether the patient has a PHR account in the system. If not, the patient is registered for a new PHR account in step 113.

The HCP next, in step 114, may scan a QR code associated with the patient account. In various embodiments, other methods may be used to access the patient account. After accessing the account in step 114, the HCP creates a record of the patient visit in step 115, completing admission of the patient.

FIG. 2B shows the process for recording the results of the various steps of the clinical examination 120 of the patient. The HCP in step 121 determines whether the patient requires medical imaging or lab testing. Then in step 122, the HCP creates a service request, and in step 123, determines whether a third-party service is required. If a third-party service is required, a service request is provided in step 124, and the third-party service provider is contacted and performs and records the results of the necessary procedure(s) in step 126. If no third-party service is required, the HCP in step 125 records the results of any in-house procedures. Finally, in step 127 the HCP enters his report on any testing and examination of the patient, including a summary of the patient's condition, description of the medical problem, diagnosis, and treatment.

FIG. 2C shows the processing 130 of a prescription. In step 131, the HCP determines whether or not the patient needs a prescription. If so, the HCP generates the prescription in step 132. In step 133, the HCP determines whether the patient needs the prescription sent to a third-party pharmacy. If so, the prescription is sent 134 to the third-party pharmacy, who in turn processes the prescription through their system. The HCP then records 135 the particulars of the prescription in the GPHR. Finally, the patient is discharged 136, and can view 137 the details of his visit in the GPHR system.

FIG. 2D illustrates the processing of an insurance claim 140 in the GPHR system. If the patient needs to make a health insurance claim 141, the claim is generated 142 by the GPHR system. Either the HCP or the patient can send the insurance claim to the third-party health insurance provider. The third-party insurance provider returns the insurance claim response either approving or denying coverage of the claim in step 143. If the claim is denied, the HCP or the patient can update 144 the claim and resubmit to the insurance provider.

It should be noted that at each step of creating and writing records in the GPHR system described herein, the process may include creating a hash and signing records, pushing them to blockchain blocks and storing the blocks' addresses in an FHIR database. Upon the signing of an EMR form, a digitized image including images of the one or more electronic signatures may be generated. The method may then proceed with generating a hash based on the digitized image. The method may also include storing the signed EMR form in an EMR data repository. The method may then proceed with storing the hash to a blockchain system. The hash can be used to verify authenticity of the signed EMR form.

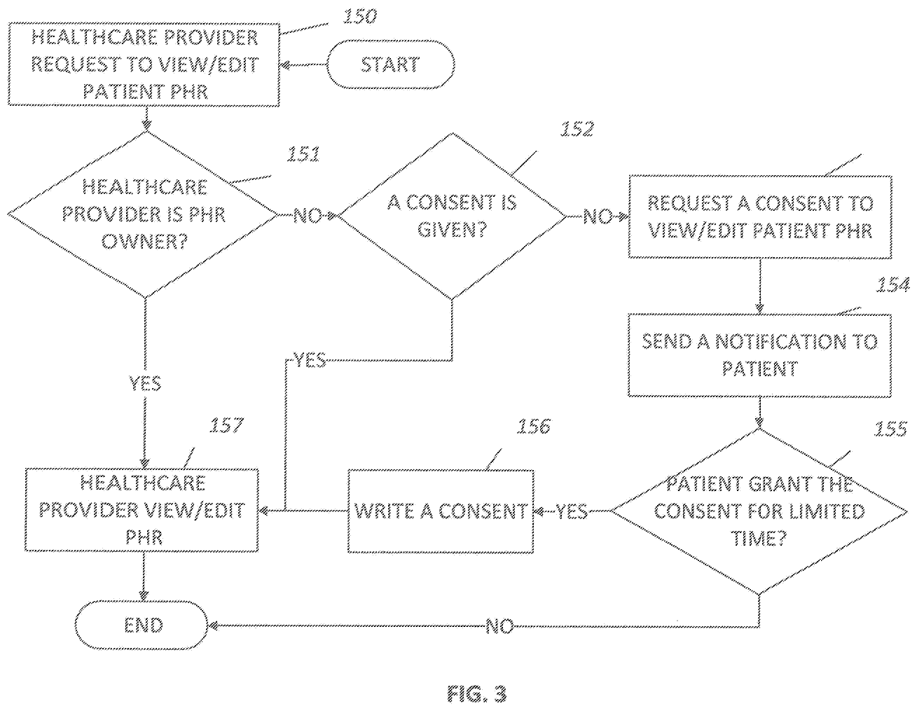

FIG. 3 illustrates the process flow that takes place when an HCP requests access to a PHR account. FIG. 3 illustrates the process flow for two situations, the HCP already having access to the account, and the alternate instance in which access is granted by the patient to the HCP. The process is initiated, step 150, by an HCP requesting to view and/or edit a PHR. When a request is received, the PHR system checks in step 151 to determine if the HCP is the owner of the subject record(s). If the HCP is not the PHR owner, the PHR system checks in step 152 to determine if proper consent has been given. If proper consent has not been given, the PHR system requests in step 153 consent from the owner of the subject record(s), and in step 154 sends notification to the owner of the record(s). If the owner of the record(s) chooses to give access to the HCP, the PHR owner grants authorization to access the record(s), typically for a limited time, in step 155. Written consent for access is generated in step 156. Finally, in step 157, the HCP views, and if necessary edits, the subject PHR.

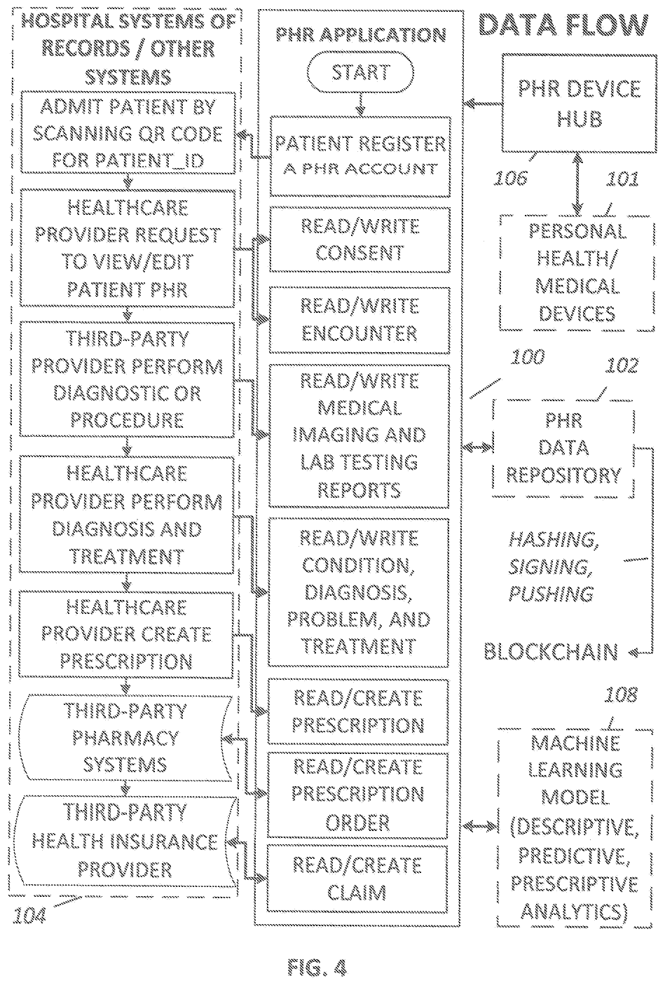

FIG. 4 presents a summary of the data flows described in FIGS. 2A-D and FIG. 3, and illustrates the data flow between the components shown in FIG. 1 that are necessary to perform the various data creation, storage, and access functions. FIG. 4 highlights the fact that the GPHR system 100 is in communication, via the PHR application module, with the PHR data repository 102. Since the data in repository 102 is protected by a hashing/blockchain system, the patient can provide secure access to his records to his HCP. The system 100 is also in secure communication with multiple hospital record keeping systems 104 (EHR, EMR, and HIS/CIS).

The system 100 also provides a convenient link to any devices the patient may utilize to create and store data in EHR's and EMR's through the PHR device hub 106. The PHR device hub 106 is of course in communication with the main GPHR system as well as personal devices 101 that are utilized by the patient.

The system 100 is also in communication with the machine learning model 108 which uses AI to generate various descriptive, predictive, and prescriptive analytic parameters.

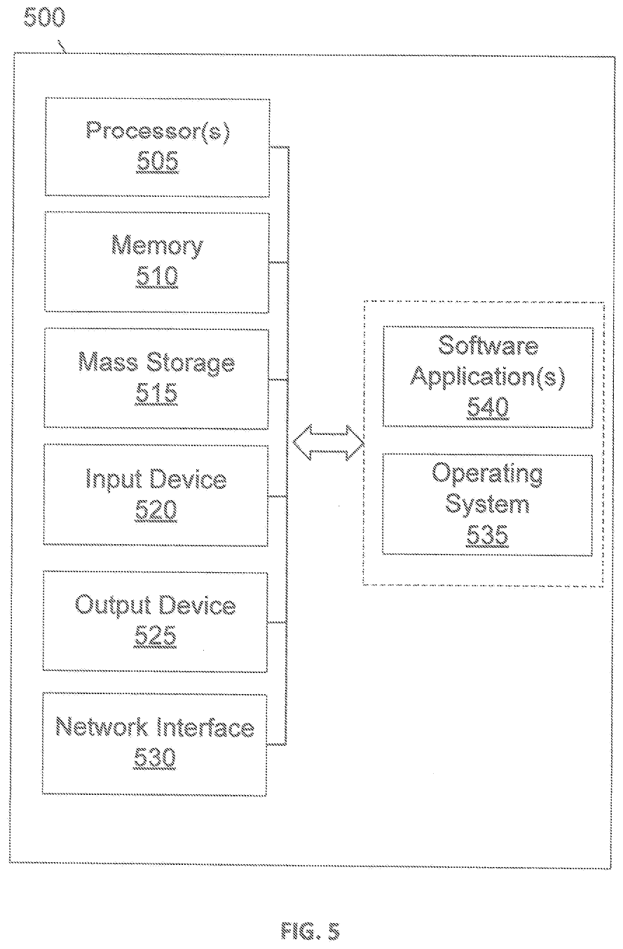

FIG. 5 is a high-level block diagram illustrating an example computer system 500, within which a set of instructions for causing the machine to perform any one or more of the methodologies discussed herein can be executed. The computer system 500 can refer to or be an integral part of one or more of a variety of types of devices, such as a general-purpose computer, desktop computer, laptop computer, tablet computer, netbook, mobile phone, smartphone, personal digital computer, smart television device, server, among others. In some embodiments, the computer system 500 can be implemented in the contexts of the likes of PHR application 100, hospital systems 104, machine learning model 108, and the associated blockchain system. Notably, FIG. 5 illustrates just one example of the computer system 500 and, in some embodiments, the computer system 500 may have fewer elements/modules than shown on FIG. 5 or more elements/modules than shown on FIG. 5.

The computer system 500 includes one or more processors 505, a memory 510, one or more storage devices 515, one or more input devices 520, one or more output devices 525, and network interface 530. One or more processors 505 are, in some examples, configured to implement functionality and/or process instructions for execution within the computer system 500. For example, the processors 505 may process instructions stored in memory 510 and/or instructions stored on storage devices 515. Such instructions may include components of an operating system 535 or software applications 540. Computer system 500 may also include one or more additional components not shown in FIG. 5, such as a housing, power supply, battery, global positioning system (GPS) receiver, and so forth.

Memory 510, according to one example, is configured to store information within the computer system 500 during operation. Memory 510, in some example embodiments, may refer to a non-transitory computer-readable storage medium or a computer-readable storage device. In some examples, memory 510 is a temporary memory, meaning that a primary purpose of memory 510 may not be long-term storage. Memory 510 may also refer to a volatile memory, meaning that memory 510 does not maintain stored contents when memory 510 is not receiving power. Examples of volatile memories include random access memories (RAM), dynamic random access memories (DRAM), static random access memories (SRAM), and other forms of volatile memories known in the art. In some examples, memory 510 is used to store program instructions for execution by the processors 505. Memory 510, in one example, is used by software (e.g., the operating system 535 or applications 540). Generally, software applications 540 refer to software applications suitable for implementing at least some operations of the methods for providing an electronic PHR system as described herein.

One or more storage devices 515 can also include one or more transitory or non-transitory computer-readable storage media and/or computer-readable storage devices. In some embodiments, storage devices 515 may be configured to store greater amounts of information than memory 510. Storage devices 515 may further be configured for long-term storage of information. In some examples, the storage devices 515 include non-volatile storage elements. Examples of such non-volatile storage elements include magnetic hard discs, optical discs, solid-state discs, flash memories, forms of electrically programmable memories (EPROM) or electrically erasable and programmable memories, and other forms of non-volatile memories known in the art.

Still referencing FIG. 5, the computer system 500 may also include one or more input devices 520. The input devices 520 may be configured to receive input from a user through tactile, audio, video, or biometric channels. Examples of input devices 520 may include a keyboard, keypad, mouse, trackball, touchscreen, touchpad, microphone, one or more video cameras, image sensors, fingerprint sensors, or any other device capable of detecting an input from a user or other source, and relaying the input to computer system 500, or components thereof.

The output devices 525, in some examples, may be configured to provide output to a user through visual or auditory channels. Output devices 525 may include a video graphics adapter card, a liquid crystal display (LCD) monitor, a light emitting diode (LED) monitor, an organic LED monitor, a sound card, a speaker, a lighting device, a LED, a projector, or any other device capable of generating output that may be intelligible to a user. Output devices 525 may also include a touchscreen, presence-sensitive display, or other input/output capable displays known in the art.

The computer system 500, in some example embodiments, also includes with network interface 530. The network interface 530 can be utilized to communicate with external devices via one or more data networks such as one or more wired, wireless, or optical networks including, for example, the Internet, intranet, LAN, WAN, cellular phone networks, Bluetooth radio, and an IEEE 802.11-based radio frequency network, among others. The network interface 530 may be a network interface card, such as an Ethernet card, an optical transceiver, a radio frequency transceiver, or any other type of device that can send and receive information.

The operating system 535 may control one or more functionalities of computer system 500 and/or components thereof. For example, the operating system 535 may interact with the applications 540 and may facilitate one or more interactions between the applications 540 and components of the computer system 500. As shown in FIG. 5, the operating system 535 may interact with or be otherwise coupled to the application(s) 540 and components thereof. In some embodiments, application(s) 540 may be included in operating system 535. In these and other examples, virtual modules, firmware, or software may be part of the applications 540.

The terminology used herein is for the purpose of describing particular embodiments only and is not intended to be limiting of the technology. As used herein, the singular forms “a”, “an” and “the” are intended to include the plural forms as well, unless the context clearly indicates otherwise. It will be further understood that the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof.

It will be understood that like or analogous elements and/or components, referred to herein, may be identified throughout the drawings with like reference characters. It will be further understood that several of the figures are merely schematic representations of the present disclosure. As such, some of the components may have been distorted from their actual scale for pictorial clarity.

In the foregoing description, for purposes of explanation and not limitation, specific details are set forth, such as particular embodiments, procedures, techniques, etc. in order to provide a thorough understanding of the present invention. However, it will be apparent to one skilled in the art that the present invention may be practiced in other embodiments that depart from these specific details.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” or “according to one embodiment” (or other phrases having similar import) at various places throughout this specification are not necessarily all referring to the same embodiment. Furthermore, the particular features, structures, or characteristics may be combined in any suitable manner in one or more embodiments. Furthermore, depending on the context of discussion herein, a singular term may include its plural forms and a plural term may include its singular form. Similarly, a hyphenated term (e.g., “on-demand”) may be occasionally interchangeably used with its non-hyphenated version (e.g., “on demand”), a capitalized entry (e.g., “Software”) may be interchangeably used with its non-capitalized version (e.g., “software”), a plural term may be indicated with or without an apostrophe (e.g., PE's or PEs), and an italicized term (e.g., “N+1”) may be interchangeably used with its non-italicized version (e.g., “N+1”). Such occasional interchangeable uses shall not be considered inconsistent with each other.

Also, some embodiments may be described in terms of “means for” performing a task or set of tasks. It will be understood that a “means for” may be expressed herein in terms of a structure, such as a processor, a memory, an I/O device such as a camera, or combinations thereof. Alternatively, the “means for” may include an algorithm that is descriptive of a function or method step, while in yet other embodiments the “means for” is expressed in terms of a mathematical formula, prose, or as a flow chart or signal diagram.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof.

While various embodiments have been described above, it should be understood that they have been presented by way of example only, and not limitation. The descriptions are not intended to limit the scope of the invention to the particular forms set forth herein. To the contrary, the present descriptions are intended to cover such alternatives, modifications, and equivalents as may be included within the spirit and scope of the invention as defined by the appended claims and otherwise appreciated by one of ordinary skill in the art. Thus, the breadth and scope of a preferred embodiment should not be limited by any of the above-described exemplary embodiments.

We claim:

1. A global personal health record (GPHR) system comprising:
at least one processor and a memory storing processor-executable codes, wherein the at least one processor is configured to implement the following operations upon executing the processor-executable codes:
retrieving and storing medical and health data from multiple data sources, the data being stored in at least one HL7 FHIR local storage when offline storage is desired, and retrieving and storing data online in HL7 FHIR server when online storage is desired;
retrieving and storing medical and health data from multiple sources;
providing a PHR device hub which provides any of a Bluetooth, Serial, and Wi-Fi connection to multiple personal health and medical devices and auto upload of data from these devices to the GPHR;
establishing a data repository which supports retrieving and storing data as HL7 FHIR records, the data repository providing application-independent access to data offline in HL7 FHIR local storage and online in HL7 FHIR server, the data repository further providing the sharing of data to specified parties with time limited authorizations, the data depository further utilizing a blockchain system using hashes for electronically signed documents, pushing the hashes to blockchain blocks and storing the addresses of the blocks in FHIR, access to individual and family data owned by the individual stored in the GPHR being controlled by the individual; and
a machine learning model which provides descriptive analytics to provide visual presentations of selected data, for example remote patient monitoring based on vital sign data from personal health/medical devices worn by the patient, predictive analytics to predict outcomes based on selected data, for example drug interaction warning based on history of treatment, diagnosis, prescription, lab results from health facilities visited by the patient, and prescriptive analytics to recommend choices for different modeling of selected data, for example medical exam or lab test recommendation based on big data of medical treatments of current medical conditions experienced by the patient.

2. The system of claim 1, wherein:
the machine learning model provides any one of visual presentations of selected data such as remote patient monitoring, predictive analytics to predict outcomes for selected courses of treatment such as analytics regarding interaction of multiple drugs, and prescriptive analytics to assist in choosing medical examination procedures and lab tests.

3. The system of claim 1, wherein:
the multiple sources include any of handwritten or voice user inputs, hospital systems of data depository records, personal devices via a PHR device hub, and insurance reimbursement and authorization systems.

4. The system of claim 1, wherein:
the processor-executable codes are configured to run on any one of Windows based laptops, Android based tablets, and iPads with Apple Pencil.

5. The system of claim 1, wherein:
the processor-executable codes are configured to run as one of a desktop application, a web application, a word application, or a mobile application.

6. The system of claim 1, wherein:
a PHR application, the data repository, and the machine learning model run on Windows devices.

7. The system of claim 6, wherein:
the PHR application, the data repository, and the machine learning model run on Windows devices as web applications.

8. The system of claim 6, wherein:
the PHR application, the data repository, and the machine learning model run on Windows devices as Windows applications.

9. The system of claim 1, wherein:
a PHR application, the data repository, and the machine learning model run on Android devices.

10. The system of claim 9, wherein:
the PHR application, the data repository, and the machine learning model run on Android devices as web applications.

11. The system of claim 9, wherein:
the PHR application, the data repository, and the machine learning model run on Android devices as Android applications.

12. The system of claim 1, wherein:
a PHR application, the data repository, and the machine learning model run on Apple devices with Apple Pencil.

13. The system of claim 12, wherein:
the PHR application, the data repository, and the machine learning model run on Apple devices with Apple Pencil as web applications.

14. The system of claim 12, wherein:
the PHR application, the data repository, and the machine learning model run on Apple devices with Apple Pencil as iOS applications.

15. The system of claim 1, wherein:
a PHR Application retrieves patient data from the hospital records systems via an application programming interface (API) for application calling, store procedures compatible with SQL databases for data sharing, and dynamic data exchanges for screen capturing.

16. The system of claim 1, wherein:
at each step of creating/writing records, the data repository hashes signed records, pushes the records to blockchain blocks and stores the addresses of the blocks in the data repository, such that the data repository can securely share records.

17. The system of claim 1, wherein:
the machine learning model uses Power BI for descriptive analytics, and Azure Machine Learning or CNTK for predictive and prescriptive analytics.

18. The system of claim 1, wherein:
the machine learning model uses Tableau for descriptive analytics, and TensorFlow for predictive and prescriptive analytics.

19. The system of claim 1, wherein:
data stored in the system is self-updated when connected to the Internet.

20. The system of claim 1, wherein:
the machine learning model self-updates its analytic models when the system is connected to the Internet.
